(19) United States
(12) Patent Application Publication
(10) Pub. No.: US 2026/0015330 A1
(43) Pub. Date: Jan. 15, 2026

(54) METHODS FOR SYNTHESIZING COMPOUNDS AND RELATED COMPOSITIONS

(71) Applicant: ENTEGRIS, INC., Billerica, MA (US)

(72) Inventors: Vagulejan Balasanthiran, Pennsburg, PA (US); Scott A. Laneman, Vernon Hills, IL (US); Kh Mahfuz Alam, North Wales, PA (US); Thomas Kermis, Lansdale, PA (US)

(21) Appl. No.: 19/267,140

(22) Filed: Jul. 11, 2025

Related U.S. Application Data
(60) Provisional application No. 63/670,471, filed on Jul. 12, 2024.

(51) Int. Cl. C07D 249/20

(52) U.S. Cl. CPC C07D 249/20

(57) ABSTRACT

Methods for synthesizing compounds are provided. A method of synthesizing a compound comprises obtaining a compound comprising an alpha halide group and a protecting group; and contacting the compound with at least one reagent sufficient to remove the protecting group (deprotect) and convert the alpha halide group to at least one of an alkenyl, an aldehyde, or any combination thereof, to obtain a reaction product at high purity. Related compositions are also provided, among other things.

### CROSS-REFERENCE TO RELATED APPLICATIONS

This application claims the benefit under 35 USC 119 of U.S. Provisional Patent Application No. 63/670,471, filed Jul. 12, 2024, the disclosure of which is hereby incorporated herein by reference in its entirety.

### FIELD

This application relates to methods for synthesizing compounds and related compositions, among other things.

### BACKGROUND

Conventional methods for synthesizing certain compounds can involve use of reagents that negatively impact the environment. In addition, conventional methods for synthesizing certain compounds can involve a multitude of steps, reducing economic feasibility and increasing complexity for manufacturing.

### SUMMARY

Some embodiments relate to a method of synthesis. In some embodiments, the method of synthesis comprises obtaining a compound of the formula:

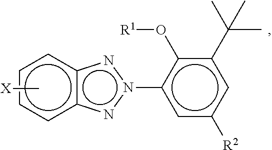

where:
X is a first halide;
R¹ is a first substituent;
R² is a second substituent. In some embodiments, the second substituent comprises a second halide. In some embodiments, the second substituent comprises an aldehyde. In some embodiments, the method of synthesis comprises contacting the compound with at least one reagent sufficient to obtain a reaction product of the formula:

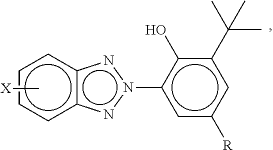

where:
X is the first halide;
R is a third substituent.

In some embodiments, the third substituent is different from the second substituent.

Some embodiments relate to a method of synthesis. In some embodiments, the method of synthesis comprises obtaining a compound of the formula:

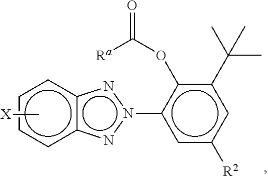

where:
X is a first halide;
R^{a} is a C₁-C₄ alkyl;
R² comprises a second halide.

In some embodiments, the method of synthesis comprises contacting the compound with at least one reagent sufficient to obtain a reaction product of the formula:

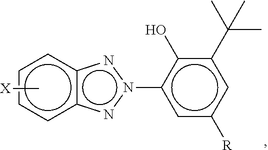

where:
X is the first halide;
R is an alkenyl.

Some embodiments relate to a method of synthesis. In some embodiments, the method of synthesis comprises obtaining a compound of the formula:

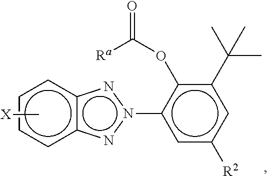

where:
X is a first halide;
R^{a} is a C₁-C₄ alkyl;
R² comprises an aldehyde.

In some embodiments, the method of synthesis comprises contacting the compound with at least one reagent sufficient to obtain a reaction product of the formula:

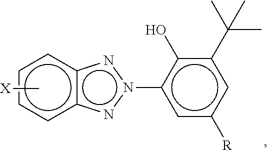

where:
X is the first halide;
R is an alkenyl.

Some embodiments relate to a composition. In some embodiments, the composition comprises a compound of the formula:

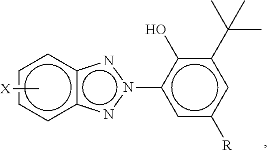

where:
X is a halide;
R is an aldehyde or an alkenyl.

In some embodiments, the compound is present in the composition at a purity of at least 97%.

### DRAWINGS

FIG. 1 is a schematic diagram of a flowchart of a method of synthesis, according to some embodiments.

FIG. 2 is a schematic diagram of a flowchart of a method of synthesis, according to some embodiments.

FIG. 3 is a schematic diagram of a flowchart of a method of synthesis, according to some embodiments.

FIG. 4 is a schematic diagram of a reaction scheme, according to some embodiments.

FIG. 5 is a schematic diagram of a reaction scheme, according to some embodiments.

### DETAILED DESCRIPTION

As used herein, the term “alkyl” refers to a hydrocarbyl having from 1 to 30 carbon atoms. The alkyl may be attached via a single bond. An alkyl having n carbon atoms may be designated as a “Cₙ alkyl.” For example, a “C₃ alkyl” may include n-propyl and isopropyl. An alkyl having a range of carbon atoms, such as 1 to 30 carbon atoms, may be designated as a C₁-C₃₀ alkyl. In some embodiments, the alkyl is linear. In some embodiments, the alkyl is branched. In some embodiments, the alkyl is substituted. In some embodiments, the alkyl is unsubstituted. In some embodiments, the alkyl comprises or is selected from the group consisting of at least one of a C₁-C₃₀ alkyl, C₁-C₂₉ alkyl, C₁-C₂₈ alkyl, C₁-C₂₇ alkyl, C₁-C₂₇ alkyl, C₁-C₂₆ alkyl, C₁-C₂₅ alkyl, C₁-C₂₄ alkyl, C₁-C₂₃ alkyl, C₁-C₂₂ alkyl, C₁-C₂₁ alkyl, C₁-C₂₀ alkyl, C₁-C₁₉ alkyl, C₁-C₁₈ alkyl, C₁-C₁₇ alkyl, C₁-C₁₆ alkyl, C₁-C₁₅ alkyl, C₁-C₁₄ alkyl, C₁-C₁₃ alkyl, C₁-C₁₂ alkyl, C₁-C₁₁ alkyl, C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, a C₂-C₃₀ alkyl, a C₃-C₃₀ alkyl, a C₄-C₃₀ alkyl, a C₅-C₃₀ alkyl, a C₆-C₃₀ alkyl, a C₇-C₃₀ alkyl, a C₈-C₃₀ alkyl, a C₉-C₃₀ alkyl, a C₁₀-C₃₀ alkyl, a C₁₁-C₃₀ alkyl, a C₁₂-C₃₀ alkyl, a C₁₃-C₃₀ alkyl, a C₁₄-C₃₀ alkyl, a C₁₅-C₃₀ alkyl, a C₁₆-C₃₀ alkyl, a C₁₇-C₃₀ alkyl, a C₁₈-C₃₀ alkyl, a C₁₉-C₃₀ alkyl, a C₂₀-C₃₀ alkyl, a C₂₁-C₃₀ alkyl, a C₂₂-C₃₀ alkyl, a C₂₃-C₃₀ alkyl, a C₂₄-C₃₀ alkyl, a C₂₅-C₃₀ alkyl, a C₂₆-C₃₀ alkyl, a C₂₇-C₃₀ alkyl, a C₂₈-C₃₀ alkyl, a C₂₉-C₃₀ alkyl, a C₂-C₁₀ alkyl, a C₃-C₁₀ alkyl, a C₄-C₁₀ alkyl, a C₅-C₁₀ alkyl, a C₆-C₁₀ alkyl, a C₇-C₁₀ alkyl, a C₈-C₁₀ alkyl, a C₂-C₉ alkyl, a C₂-C₈ alkyl, a C₂-C₇ alkyl, a C₂-C₆ alkyl, a C₂-C₅ alkyl, a C₃-C₅ alkyl, or any combination thereof. In some embodiments, the alkyl comprises or is selected from the group consisting of at least one of methyl, ethyl, n-propyl, 1-methylethyl (iso-propyl), n-butyl, iso-butyl, sec-butyl, n-pentyl, 1,1-dimethylethyl (t-butyl), n-pentyl, iso-pentyl, n-hexyl, isohexyl, 3-methylhexyl, 2-methylhexyl, heptyl, octyl, nonyl, decyl, dodecyl, octadecyl, or any combination thereof. In some embodiments, the term “alkyl” refers generally to alkyls, alkenyls, alkynyls, and/or cycloalkyls.

As used herein, the term “alkenyl” refers to a hydrocarbyl having from 1 to 30 carbon atoms and at least one carbon-carbon double bond. In some embodiments, the alkenyl comprises or is selected from the group consisting of at least one of a C₁-C₃₀ alkenyl, C₁-C₂₉ alkenyl, C₁-C₂₈ alkenyl, C₁-C₂₇ alkenyl, C₁-C₂₇ alkenyl, C₁-C₂₆ alkenyl, C₁-C₂₅ alkenyl, C₁-C₂₄ alkenyl, C₁-C₂₃ alkenyl, C₁-C₂₂ alkenyl, C₁-C₂₁ alkenyl, C₁-C₂₀ alkenyl, C₁-C₁₉ alkenyl, C₁-C₁₈ alkenyl, C₁-C₁₇ alkenyl, C₁-C₁₆ alkenyl, C₁-C₁₅ alkenyl, C₁-C₁₄ alkenyl, C₁-C₁₃ alkenyl, C₁-C₁₂ alkenyl, C₁-C₁₁ alkenyl, C₁-C₁₀ alkenyl, a C₁-C₉ alkenyl, a C₁-C₈ alkenyl, a C₁-C₇ alkenyl, a C₁-C₆ alkenyl, a C₁-C₅ alkenyl, a C₁-C₄ alkenyl, a C₁-C₃ alkenyl, a C₁-C₂ alkenyl, a C₂-C₃₀ alkenyl, a C₃-C₃₀ alkenyl, a C₄-C₃₀ alkenyl, a C₅-C₃₀ alkenyl, a C₆-C₃₀ alkenyl, a C₇-C₃₀ alkenyl, a C₈-C₃₀ alkenyl, a C₉-C₃₀ alkenyl, a C₁₀-C₃₀ alkenyl, a C₁₁-C₃₀ alkenyl, a C₁₂-C₃₀ alkenyl, a C₁₃-C₃₀ alkenyl, a C₁₄-C₃₀ alkenyl, a C₁₅-C₃₀ alkenyl, a C₁₆-C₃₀ alkenyl, a C₁₇-C₃₀ alkenyl, a C₁₈-C₃₀ alkenyl, a C₁₉-C₃₀ alkenyl, a C₂₀-C₃₀ alkenyl, a C₂₁-C₃₀ alkenyl, a C₂₂-C₃₀ alkenyl, a C₂₃-C₃₀ alkenyl, a C₂₄-C₃₀ alkenyl, a C₂₅-C₃₀ alkenyl, a C₂₆-C₃₀ alkenyl, a C₂₇-C₃₀ alkenyl, a C₂₈-C₃₀ alkenyl, a C₂₉-C₃₀ alkenyl, a C₂-C₁₀ alkenyl, a C₃-C₁₀ alkenyl, a C₄-C₁₀ alkenyl, a C₅-C₁₀ alkenyl, a C₆-C₁₀ alkenyl, a C₇-C₁₀ alkenyl, a C₈-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₃-C₅ alkenyl, or any combination thereof. Examples of alkenyl groups include, without limitation, at least one of vinyl, allyl, 1-methylvinyl, 1-propenyl, 1-butenyl, 2-butenyl, 3-butenyl, 1,3-butadienyl, 2-methyl-1-propenyl, 2-methyl-2-propenyl, 1-pentenyl, 2-pentenyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 3-heptenyl, 1-octenyl, 1,3-octadienyl, 1-nonenyl, 2-nonenyl, 3-nonenyl, 1-decenyl, 3-decenyl, 1-undecenyl, oleyl, linoleyl, linolenyl, or any combination thereof.

As used herein, the term “alkynyl” refers to a hydrocarbyl having from 1 to 30 carbon atoms and at least one carbon-carbon triple bond. In some embodiments, the alkynyl comprises or is selected from the group consisting of at least one of a C₁-C₃₀ alkynyl, C₁-C₂₉ alkynyl, C₁-C₂₈ alkynyl, C₁-C₂₇ alkynyl, C₁-C₂₇ alkynyl, C₁-C₂₆ alkynyl, C₁-C₂₅ alkynyl, C₁-C₂₄ alkynyl, C₁-C₂₃ alkynyl, C₁-C₂₂ alkynyl, C₁-C₂₁ alkynyl, C₁-C₂₀ alkynyl, C₁-C₁₉ alkynyl, C₁-C₁₈ alkynyl, C₁-C₁₇ alkynyl, C₁-C₁₆ alkynyl, C₁-C₁₅ alkynyl, C₁-C₁₄ alkynyl, C₁-C₁₃ alkynyl, C₁-C₁₂ alkynyl, C₁-C₁₁ alkynyl, C₁-C₁₀ alkynyl, a C₁-C₉ alkynyl, a C₁-C₈ alkynyl, a C₁-C₇ alkynyl, a C₁-C₆ alkynyl, a C₁-C₅ alkynyl, a C₁-C₄ alkynyl, a C₁-C₃ alkynyl, a C₁-C₂ alkynyl, a C₂-C₃₀ alkynyl, a C₃-C₃₀ alkynyl, a C₄-C₃₀ alkynyl, a C₅-C₃₀ alkynyl, a C₆-C₃₀ alkynyl, a C₇-C₃₀ alkynyl, a C₈-C₃₀ alkynyl, a C₉-C₃₀ alkynyl, a C₁₀-C₃₀ alkynyl, a C₁₁-C₃₀ alkynyl, a C₁₂-C₃₀ alkynyl, a C₁₃-C₃₀ alkynyl, a C₁₄-C₃₀ alkynyl, a C₁₅-C₃₀ alkynyl, a C₁₆-C₃₀ alkynyl, a C₁₇-C₃₀ alkynyl, a C₁₈-C₃₀ alkynyl, a C₁₉-C₃₀ alkynyl, a C₂₀-C₃₀ alkynyl, a C₂₁-C₃₀ alkynyl, a C₂₂-C₃₀ alkynyl, a C₂₃-C₃₀ alkynyl, a C₂₄-C₃₀ alkynyl, a C₂₅-C₃₀ alkynyl, a C₂₆-C₃₀ alkynyl, a C₂₇-C₃₀ alkynyl, a C₂₈-C₃₀ alkynyl, a C₂₉-C₃₀ alkynyl, a C₂-C₁₀ alkynyl, a C₃-C₁₀ alkynyl, a C₄-C₁₀ alkynyl, a C₅-C₁₀ alkynyl, a C₆-C₁₀ alkynyl, a C₇-C₁₀ alkynyl, a C₈-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₃-C₅ alkynyl, or any combination thereof. Examples of alkynyl groups include, without limitation, at least one of ethynyl, propynyl, n-butynyl, n-pentynyl, 3-methyl-1-butynyl, n-hexynyl, methyl-pentynyl, or any combination thereof.

As used herein, the term “haloalkyl” refers to an alkyl as defined here, wherein at least one of the hydrogen atoms of the alkyl is replaced with a halide as defined herein. In some embodiments, the haloalkyl comprises a fluoroalkyl. In some embodiments, the fluoroalkyl comprises at least one of —CH₂CF₃, —CH(CF₃)₂, —CH₂F, —CH₂CH₂F, —CF₃, —CF₂CF₃, or any combination thereof.

As used herein, the term “halide” refers to a —Cl, —Br, —I, or —F.

As used herein, the term “vinyl” refers to —CH═CH₂.

As used herein, the term “acetyl” refers to —C(═O)CH₃.

As used herein, the term “acetoxy” refers to —OC(═O)CH₃.

As used herein, the term “metal” refers to at least one of an alkali metal, an alkaline earth metal, a transition metal, a post-transition metal, or any combination thereof. In some embodiments, the metal comprises a metal cation. In some embodiments, the metal cation comprises at least one of a lithium cation, a sodium cation, a potassium cation, a rubidium cation, a cesium cation, a francium cation, a beryllium cation, a magnesium cation, a calcium cation, a strontium cation, a barium cation, a radium cation, a scandium cation, a titanium cation, a vanadium cation, a chromium cation, a manganese cation, an iron cation, a cobalt cation, a nickel cation, a copper cation, a zinc cation, a yttrium cation, a zirconium cation, a niobium cation, a molybdenum cation, a technetium cation, a ruthenium cation, a rhodium cation, a palladium cation, a silver cation, a cadmium cation, a hafnium cation, a tantalum cation, a tungsten cation, a rhenium cation, an osmium cation, an iridium cation, a platinum cation, a gold cation, a mercury cation, an aluminum cation, a gallium cation, an indium cation, tin cation, a thallum cation, a lead cation, a bismuth cation, a polonium cation, or any combination thereof. The charge(s) of the metal cations are known and, for simplicity, thus are not repeated here; however, it will be appreciated that the metal cations can have any known charge.

The synthesis of various compounds, according to conventional methods, can require a multitude of complex steps. For example, the synthesis of compounds such as, 2-(tert-Butyl)-6-(5-chloro-2H-benzo[d][1,2,3]triazol-2-yl)-4-vinylphenol (UVAM) and other benzotriazole compounds with vinyl substituents, can involve nine or more steps, for example, thirteen steps, or more. In addition, conventional methods require the use of compounds classified as toxic, corrosive, or generally not environmentally friendly, including, for example and without limitation, at least one of CCl₄, AgNO₃, bromine, picric acid, or any combination thereof, and the like.

Various method for synthesizing compounds, and related compositions and related methods, are provided herein. The methods disclosed herein overcome at least some of the challenges of conventional methods of synthesis. In some embodiments, for example, methods are provided for synthesizing UVAM and other benzotriazole compound with vinyl substituents, as well as vinyl phenols, such as, for example and without limitation, 2,6-Di-tert-butyl-4-vinylphenol, that require fewer reaction steps than conventional methods. In some embodiments, methods are provided in which a halide-containing substituent (e.g., an alpha halide species, such as, an alpha bromo species) and/or an aldehyde substituent is eliminated to form an alkenyl (e.g., a vinyl group), while also deprotecting or removing an acetyl group (or variation thereof) to form a hydroxyl group, in a single step. For example, in some embodiments, methods are provided for elimination and deprotection of a compound in a single pot, using less costly and more environmentally friendly reagents, among other things. In some embodiments, a reaction product is obtained in a single step when one or more reagents is capable of resulting in two or more transformations. In some embodiments, a reaction product is obtained in a single step when one or more reagents is capable of resulting in two or more transformations, without any intermediate isolation step. In some embodiments, a transformation refers to any chemical modification, substitution, or otherwise interaction between chemical species, including, for example and without limitation, the elimination and deprotection transformations discussed herein, among others.

Methods for synthesizing compounds are provided. A method of synthesizing a compound comprises obtaining a compound comprising an alpha halide group and/or an aldehyde group, and a protecting group; and contacting the compound with at least one reagent sufficient to remove the protecting group (deprotect) and convert the alpha halide group to at least one of an alkenyl, an aldehyde, or any combination thereof, to obtain a reaction product at high purity. Related compositions are also provided, among other things.

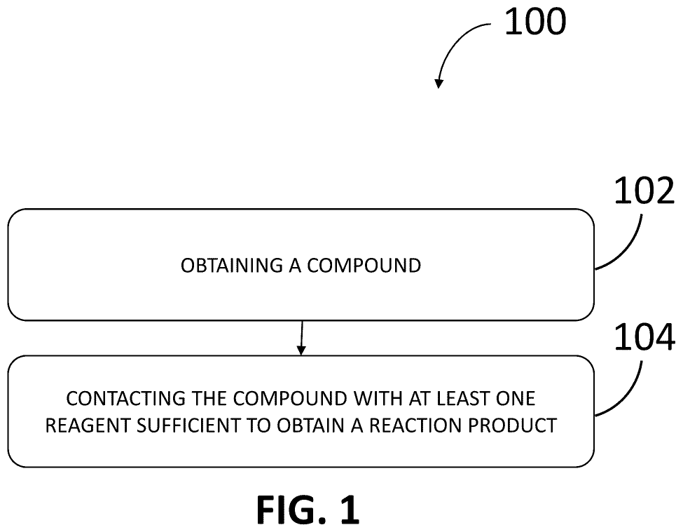

FIG. 1 is a schematic diagram of a flowchart of a method of synthesis 100, according to some embodiments. As shown in FIG. 1, in some embodiments, the method of synthesis 100 comprises one or more of the following steps: obtaining 102 a compound;

and contacting 104 the compound with at least one reagent sufficient to obtain a reaction product.

At step 102, in some embodiments, the method of synthesis 100 comprises obtaining 102 a compound.

In some embodiments, the compound comprises a compound of the formula:

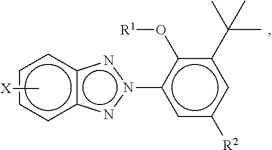

where:
X is a first halide;
R¹ is a first substituent; and
R² is a second substituent.

In some embodiments, the second substituent comprises a second halide. In some embodiments, the second substituent comprises an aldehyde.

In some embodiments, R¹ is a substituent of the formula:

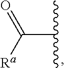

where:
R^{a} is an alkyl.

In some embodiments, R¹ is an acetyl.

In some embodiments, R² is a substituent of the formula:

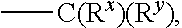

where:
R^{x} is a halide, an alkyl, or a haloalkyl; and
R^{y} is a halide, an alkyl, or a haloalkyl.

In some embodiments, R^{x} and R^{y} are different.

In some embodiments, R^{x} is a halide and R^{y} is an alkyl. In some embodiments, R^{x} is an alkyl and R^{y} is a halide. In some embodiments, R^{x} and R^{y} are alkyls. In some embodiments, R^{x} and R^{y} are halides.

In some embodiments, R² is a substituent of the formula:

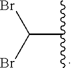 [SMILES: *C(Br)Br]

In some embodiments, R² is a substituent of the formula:

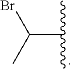 [SMILES: *C(C)Br]

In some embodiments, R² is an aldehyde (e.g., —CHO).

At step 104, in some embodiments, the method of synthesis 100 comprises contacting the compound with at least one reagent sufficient to obtain a reaction product.

In some embodiments, the at least one reagent comprises at least one of a dimethylformamide (DMF), a water, a metal tert-butoxide, a tetrahydrofuran (THF), a PPh₃(CH₃)Br compound, or any combination thereof. In some embodiments, the at least one reagent comprises a potassium tert-butoxide and a tetrahydrofuran. In some embodiments, the at least one reagent comprises a dimethyl formamide and a water. In some embodiments, the at least one reagent comprises a potassium tert-butoxide compound, a tetrahydrofuran, and a PPh₃(CH₃)Br compound.

In some embodiments, the reaction product is a compound of the formula:

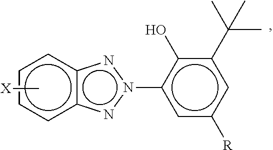

where:
X is the first halide;
R is a third substituent.

In some embodiments, the third substituent is different from the second substituent. In some embodiments, the third substituent is not an alkynyl.

In some embodiments, the third substituent is an alkenyl.

In some embodiments, the contacting proceeds in a single step. In some embodiments, the contacting comprises bringing the compound and the at least one reagent into immediate or close proximity. In some embodiments, the contacting comprises bringing the compound and the at least one reagent into direct physical contact. In some embodiments, the contacting is performed in a presence of a solvent. In some embodiments, the contacting comprises mixing the compound and the at least one reagent. In some embodiments, the contacting comprises stirring the compound and the at least one reagent. In some embodiments, the contacting comprises agitating the compound and the at least one reagent. In some embodiments, the contacting comprises adding the at least one reagent to a flask containing the compound. In some embodiments, the contacting comprises dissolving the compound in the at least one reagent. In some embodiments, the contacting comprises heating at least one of the compound, the at least one reagent, or any combination thereof. In some embodiments, the contacting comprises cooling at least one of the compound, the at least one reagent, or any combination thereof. In some embodiments, the contacting does not comprise a step in which a compound and/or a reaction product is at least partially isolated.

In some embodiments, the contacting is performed in a presence of a base. In some embodiments, the contacting is performed in an excess of a base. In some embodiments, the contacting is performed in an excess of a base compound sufficient to result in deprotection of the phenol, or hydroxyl group, by removal of the acetyl group. In some embodiments, the contacting is performed using an excess of a base compound to result in deprotection of the phenol, or hydroxyl group, by removal of the acetyl group. In some embodiments, the contacting comprises a debromination step in which the substituent is converted to an alkenyl, such as, a vinyl, or an aldehyde. A non-limiting example of a base is an organic base. In some embodiments, the method does not comprise an inorganic base. In some embodiments, the contacting is performed at about room temperature (e.g., 20° C. to 30° C.). In some embodiments, the contacting is performed sufficient such that the halide, X, is not hydrolyzed.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 0° C. to 40° C. In some embodiments, the contacting proceeds at a temperature of 10° C. to 150° C.

In some embodiments, the reaction product is a compound of the formula:

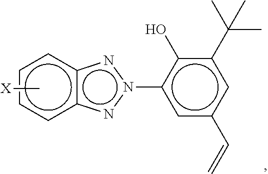

where:
X is Cl, Br, F, or I.

In some embodiments, the steps of the method 100 do not involve use of certain compounds, such as those compounds not considered to be environmentally friendly. In some embodiments, the method does not comprise In some embodiments, the method does not comprise CCl₄. In some embodiments, the method does not comprise AgNO₃.

In some embodiments, the reaction product has a purity of at least 95%, at least 96%, at least 97%, at least 98%, at least 99%, at least 99.9%, at least 99.99%, at least 99.999%. For example, in some embodiments, the reaction product has a purity of 95% to 99.9999%, 95% to 99.999%, 95% to 99.99%, 95% to 99.9%, 95% to 99%, 95% to 98%, 95% to 97%, 95% to 96%, 96% to 99.9999%, 97% to 99.9999%, 98% to 99.9999%, 99% to 99.9999%, 99.9% to 99.9999%, 99.99% to 99.9999%, or 99.999% to 99.9999%, or any range or subrange between 95% and 99.9999%.

In some embodiments, the second reaction product has a purity of at least 95%, at least 96%, at least 97%, at least 98%, at least 99%, at least 99.9%, at least 99.99%, at least 99.999%. For example, in some embodiments, the second reaction product has a purity of 95% to 99.9999%, 95% to 99.999%, 95% to 99.99%, 95% to 99.9%, 95% to 99%, 95% to 98%, 95% to 97%, 95% to 96%, 96% to 99.9999%, 97% to 99.9999%, 98% to 99.9999%, 99% to 99.9999%, 99.9% to 99.9999%, 99.99% to 99.9999%, or 99.999% to 99.9999%, or any range or subrange between 95% and 99.9999%.

In some embodiments, the method of synthesis 100 comprises obtaining a compound of the formula:

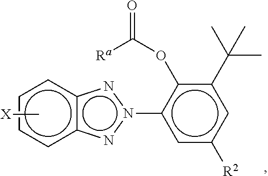

where:
X is a first halide;
R^{a} is an alkyl;
R² comprises a second halide; and
contacting the compound with at least one reagent sufficient to obtain a reaction product of the formula:

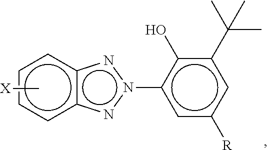

where:
X is the first halide;
R is an alkenyl.

In some embodiments, the at least one reagent comprises at least one of a metal tert-butoxide compound, a tetrahydrofuran, or any combination thereof.

In some embodiments, the method of synthesis 100 comprises obtaining a compound of the formula:

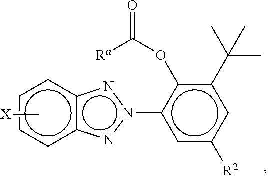

where:
X is a first halide;
R^{a} is an alkyl;
R² comprises an aldehyde; and
contacting the compound with at least one reagent sufficient to obtain a reaction product of the formula:

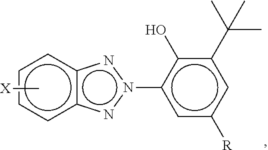

where:
X is the first halide;
R is an alkenyl.

In some embodiments, the at least one reagent comprises at least one of a metal tert-butoxide, a PPh₃(CH₃)Br compound, a tetrahydrofuran, or any combination thereof.

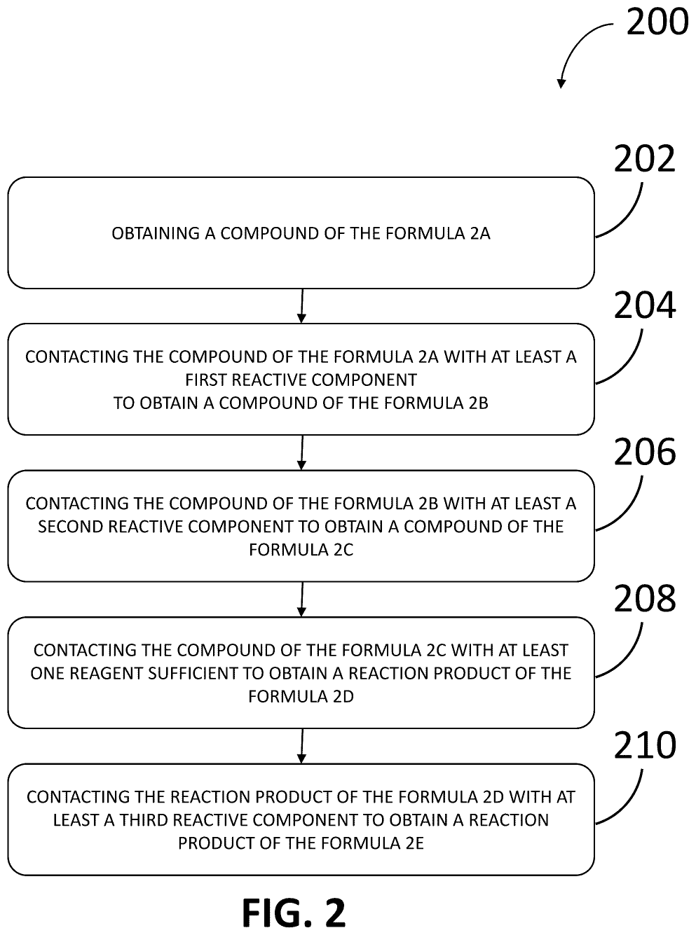

FIG. 2 is a schematic diagram of a flowchart of a method of synthesis 200, according to some embodiments. As shown in FIG. 2, in some embodiments, the method of synthesis 200 comprises one or more of the following steps: obtaining 202 a compound of the formula 2A; contacting 204 the compound of the formula 2A with at least a first reactive component to obtain a compound of the formula 2B; contacting 206 the compound of the formula 2B with at least a second reactive component to obtain a compound of the formula 2C; contacting 208 the compound of the formula 2C with at least one reagent sufficient to obtain a reaction product of the formula 2D; contacting 210 the reaction product of the formula 2D with at least a third reactive component to obtain a reaction product of the formula 2E.

At step 202, in some embodiments, the method of synthesis 200 comprises obtaining a compound of the formula 2A.

In some embodiments, the compound of the formula 2A is a compound of the formula:

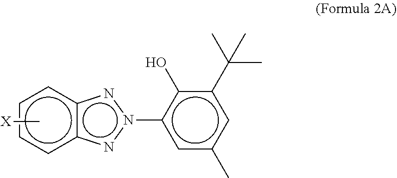

where:
X is a halide.

At step 204, in some embodiments, the method of synthesis 200 comprises contacting 204 the compound of formula 2A with at least a first reactive component to obtain a compound of the formula 2B.

In some embodiments, the compound of the formula 2B is a compound of the formula:

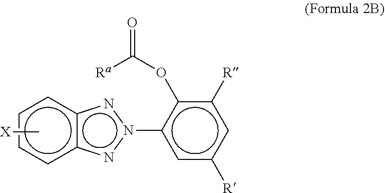

where:
X is a halide;
R^{a} is an alkyl;
R″ is an alkyl;
R′ is an alkyl.

In some embodiments, the compound of formula 2B is a compound of the formula:

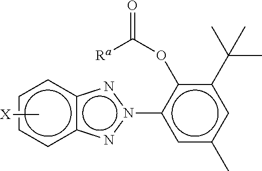

where:
R^{a} is an alkyl; and
X is a halide.

In some embodiments, the contacting comprises bringing the compound of formula 2A and at least the first reactive component into immediate or close proximity. In some embodiments, the contacting comprises brining the compound of formula 2A and at least the first reactive component into direct physical contact. In some embodiments, the contacting is performed in a presence of a solvent. In some embodiments, the contacting comprises mixing the compound of formula 2A and at least the first reactive component. In some embodiments, the contacting comprises stirring the compound of formula 2A and at least the first reactive component. In some embodiments, the contacting comprises agitating the compound of formula 2A and at least the first reactive component. In some embodiments, the contacting comprises adding at least the first reactive component to a flask containing the compound of formula 2A. In some embodiments, the contacting comprises dissolving the compound of formula 2A in at least the first reactive component. In some embodiments, the contacting comprises heating at least one of the compound of formula 2A, the first reactive component, or any combination thereof. In some embodiments, the contacting comprises cooling at least one of the compound of formula 2A, the first reactive component, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the first reactive component comprises at least one of an anhydride compound, an acid compound, or any combination thereof. In some embodiments, the first reactive component comprises at least one of acetic anhydride, H₂SO₄, or any combination thereof.

At step 206, in some embodiments, the method of synthesis 200 comprises contacting the compound of the formula 2B with at least a second reactive component to obtain a compound of the formula 2C.

In some embodiments, the compound of the formula 2C is a compound of the formula:

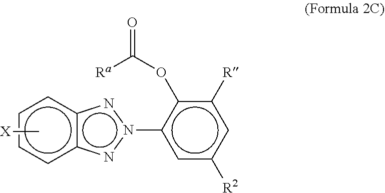

where:
X is a first halide;
R^{a} is an alkyl;
R″ is an alkyl;
R² comprises a second halide.

In some embodiments, the compound of the formula 2C is a compound of the formula:

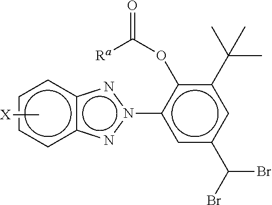

where:
X is a halide;
R^{a} is an alkyl.

In some embodiments, the contacting comprises bringing the compound of formula 2B and at least the second reactive component into immediate or close proximity. In some embodiments, the contacting comprises brining the compound of formula 2B and at least the second reactive component into direct physical contact. In some embodiments, the contacting is performed in a presence of a solvent. In some embodiments, the contacting comprises dissolving the compound of formula 2B in a solvent, such as, for example and without limitation, dichloromethane (DCM). In some embodiments, the contacting comprises mixing the compound of formula 2B and at least the second reactive component. In some embodiments, the contacting comprises stirring the compound of formula 2B and at least the second reactive component. In some embodiments, the contacting comprises agitating the compound of formula 2B and at least the second reactive component. In some embodiments, the contacting comprises adding at least the second reactive component to a flask containing the compound of formula 2B. In some embodiments, the contacting comprises dissolving the compound of formula 2B in at least the second reactive component. In some embodiments, the contacting comprises heating at least one of the compound of formula 2B, the second reactive component, or any combination thereof. In some embodiments, the contacting comprises cooling at least one of the compound of formula 2B, the second reactive component, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the second reactive component comprises at least one of a N-bromosuccinimide (NBS), a chlorobenzene, an azobisisobutyronitrile (AIBN), or any combination thereof.

At step 208, in some embodiments, the method of synthesis 200 comprises contacting the compound of the formula 2C with at least one reagent sufficient to obtain a reaction product of the formula 2D.

In some embodiments, the reaction product of the formula 2D is a compound of the formula:

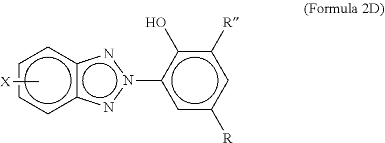

where:
X is a halide;
R″ is an alkyl;
R is an aldehyde.

In some embodiments, the reaction product of the formula 2D is a compound of the formula:

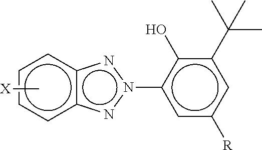

where:
X is a halide;
R is an aldehyde.

In some embodiments, the contacting comprises bringing the compound of formula 2C and the at least one reagent into immediate or close proximity. In some embodiments, the contacting comprises brining the compound of formula 2C and the at least one reagent into direct physical contact. In some embodiments, the contacting is performed in a presence of a solvent. In some embodiments, the contacting comprises dissolving the compound of formula 2C in a solvent. In some embodiments, the contacting comprises mixing the compound of formula 2C and the at least one reagent. In some embodiments, the contacting comprises stirring the compound of formula 2C and the at least one reagent. In some embodiments, the contacting comprises agitating the compound of formula 2C and the at least one reagent. In some embodiments, the contacting comprises adding the at least one reagent to a flask containing the compound of formula 2C. In some embodiments, the contacting comprises dissolving the compound of formula 2C in the at least one reagent. In some embodiments, the contacting comprises heating at least one of the compound of formula 2C, the at least one reagent, or any combination thereof. In some embodiments, the contacting comprises cooling at least one of the compound of formula 2C, the at least one reagent, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the at least one reagent comprises at least one of a dimethylformamide and a water.

At step 210, in some embodiments, the method of synthesis 200 comprises contacting the reaction product of the formula 2D with at least a third reactive component to obtain a reaction product of the formula 2E.

In some embodiments, the reaction product of the formula 2E is a compound of the formula:

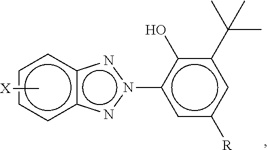

where:
X is a halide; and
R is an alkenyl.

In some embodiments, the contacting proceeds in a single step. In some embodiments, the contacting comprises bringing the compound of formula 2D and at least the third reactive component into immediate or close proximity. In some embodiments, the contacting comprises brining the compound of formula 2D and at least the third reactive component into direct physical contact. In some embodiments, the contacting is performed in a presence of a solvent. In some embodiments, the contacting comprises dissolving the compound of formula 2D in a solvent. In some embodiments, the contacting comprises mixing the compound of formula 2D and at least the third reactive component. In some embodiments, the contacting comprises stirring the compound of formula 2D and at least the third reactive component. In some embodiments, the contacting comprises agitating the compound of formula 2D and at least the third reactive component. In some embodiments, the contacting comprises adding at least the third reactive component to a flask containing the compound of formula 2D. In some embodiments, the contacting comprises dissolving the compound of formula 2D in at least the third reactive component. In some embodiments, the contacting comprises heating at least one of the compound of formula 2D, the third reactive component, or any combination thereof. In some embodiments, the contacting comprises cooling at least one of the compound of formula 2D, the third reactive component, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the third reactive component comprises at least one of a metal tert-butoxide compound, a PPh₃(CH₃)Br compound, or any combination thereof.

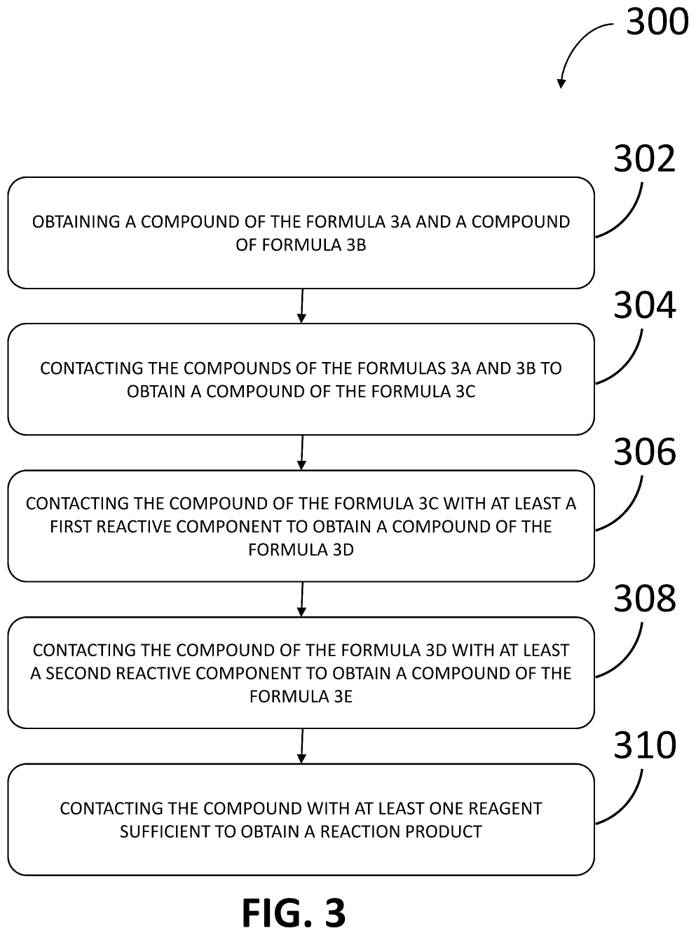

FIG. 3 is a schematic diagram of a flowchart of a method of synthesis 300, according to some embodiments. As shown in FIG. 3, in some embodiments, the method of synthesis 300 comprises one or more of the following steps: obtaining 302 a compound of the formula 3A and a compound of the formula 3B; contacting 304 the compounds of the formulas 3A and 3B to obtain a compound of the formula 3C; contacting 306 the compound of the formula 3C with at least a first reactive component to obtain a compound of the formula 3D; contacting 308 the compound of the formula 3D with at least a second reactive component to obtain a compound of the formula 3E; and contacting 310 the compound with at least one reagent sufficient to obtain a reaction product.

At step 302, in some embodiments, the method of synthesis 300 comprises obtaining 302 a compound of the formula 3A and a compound of the formula 3B.

In some embodiments, the compound of the formula 3A is a compound of the formula:

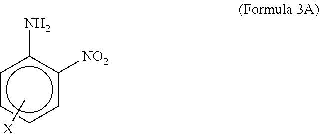

where:
X is a halide.

In some embodiments, the compound of the formula 3B is a compound of the formula:

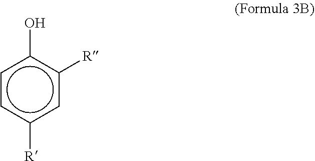

where:
R′ is an alkyl;
R″ is an alkyl.

At step 304, in some embodiments, the method of synthesis 300 comprises contacting the compounds of the formulas 3A and 3B under conditions sufficient to obtain a compound of the formula 3C.

In some embodiments, the compound of the formula 3C is a compound of the formula:

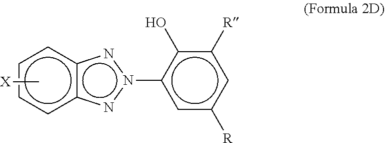

where:
X is a halide;
R′ is an alkyl; and
R″ is an alkyl.

In some embodiments, the contacting comprises bringing the compounds of formulas 3A and 3B into immediate or close proximity. In some embodiments, the contacting comprises brining the compounds of formulas 3A and 3B into direct physical contact. In some embodiments, the contacting is performed in a presence of a solvent. In some embodiments, the contacting comprises dissolving the compounds of formulas 3A and 3B in a solvent. In some embodiments, the contacting comprises mixing the compounds of formulas 3A and 3B. In some embodiments, the contacting comprises stirring the compounds of formulas 3A and 3B. In some embodiments, the contacting comprises agitating the compounds of formulas 3A and 3B. In some embodiments, the contacting comprises adding at least the compounds of formulas 3A and 3B to a flask containing the compound of formula 2D. In some embodiments, the contacting comprises heating at least one of the compound of formula 3A, the compound of formula 3B, or any combination thereof. In some embodiments, the contacting comprises cooling at least one of the compound of formula 3A, the compound of formula 3B, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

At step 306, in some embodiments, the method of synthesis 300 comprises contacting the compound of the formula 3C with at least a first reactive component to obtain a compound of the formula 3D.

In some embodiments, the compound of the formula 3D is a compound of the formula:

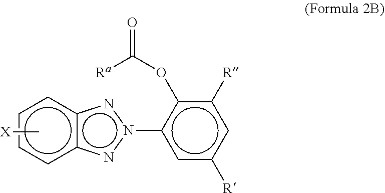

where:
X is a halide;
R^{a} is an alkyl;
R′ is an alkyl;
R″ is an alkyl.

In some embodiments, the contacting comprises bringing the compound of formula 3C and at least the first reactive component into immediate or close proximity. In some embodiments, the contacting comprises brining the compound of formula 3C and at least the first reactive component into direct physical contact. In some embodiments, the contacting is performed in a presence of a solvent. In some embodiments, the contacting comprises mixing the compound of formula 3C and at least the first reactive component. In some embodiments, the contacting comprises stirring the compound of formula 3C and at least the first reactive component. In some embodiments, the contacting comprises agitating the compound of formula 3C and at least the first reactive component. In some embodiments, the contacting comprises adding at least the first reactive component to a flask containing the compound of formula 3C. In some embodiments, the contacting comprises dissolving the compound of formula 3C in at least the first reactive component. In some embodiments, the contacting comprises heating at least one of the compound of formula 3C, the first reactive component, or any combination thereof. In some embodiments, the contacting comprises cooling at least one of the compound of formula 3C, the first reactive component, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the first reactive component comprises at least one of an anhydride compound, an acid compound, or any combination thereof. In some embodiments, the first reactive component comprises at least one of acetic anhydride, H₂SO₄, or any combination thereof.

At step 308, in some embodiments, the method of synthesis 300 comprises contacting the compound of the formula 3D with at least a second reactive component to obtain a compound of the formula 3E.

In some embodiments, the compound of the formula 3E is a compound of the formula:

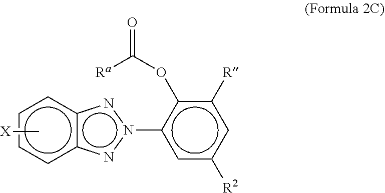

where:
X is a first halide;
R^{a} is an alkyl;
R″ is an alkyl;
R² comprises a second halide.

In some embodiments, the contacting comprises bringing the compound of formula 3D and at least the second reactive component into immediate or close proximity. In some embodiments, the contacting comprises brining the compound of formula 3D and at least the second reactive component into direct physical contact. In some embodiments, the contacting is performed in a presence of a solvent. In some embodiments, the contacting comprises dissolving the compound of formula 3D in a solvent, such as, for example and without limitation, DCM. In some embodiments, the contacting comprises mixing the compound of formula 3D and at least the second reactive component. In some embodiments, the contacting comprises stirring the compound of formula 3D and at least the second reactive component. In some embodiments, the contacting comprises agitating the compound of formula 3D and at least the second reactive component. In some embodiments, the contacting comprises adding at least the second reactive component to a flask containing the compound of formula 3D. In some embodiments, the contacting comprises dissolving the compound of formula 3D in at least the second reactive component. In some embodiments, the contacting comprises heating at least one of the compound of formula 3D, the second reactive component, or any combination thereof. In some embodiments, the contacting comprises cooling at least one of the compound of formula 3D, the second reactive component, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

At step 310, in some embodiments, the method of synthesis 300 comprises contacting the compound of formula 3E with at least one reagent sufficient to obtain a reaction product.

In some embodiments, the reaction product is a compound of the formula:

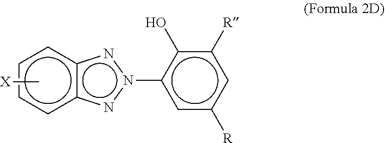

where:
X is the first halide;
R″ is an alkyl;
R is an alkenyl.

In some embodiments, the reaction product is a compound of the formula:

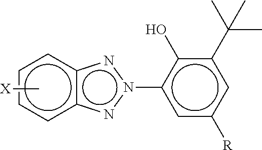

where:
X is a halide;
R is an alkenyl.

In some embodiments, the contacting comprises bringing the compound of formula 3E and the at least one reagent into immediate or close proximity. In some embodiments, the contacting comprises brining the compound of formula 3E and the at least one reagent into direct physical contact. In some embodiments, the contacting is performed in a presence of a solvent. In some embodiments, the contacting comprises dissolving the compound of formula 3E in a solvent. In some embodiments, the contacting comprises mixing the compound of formula 3E and the at least one reagent. In some embodiments, the contacting comprises stirring the compound of formula 3E and the at least one reagent. In some embodiments, the contacting comprises agitating the compound of formula 3E and the at least one reagent. In some embodiments, the contacting comprises adding the at least one reagent to a flask containing the compound of formula 3E. In some embodiments, the contacting comprises dissolving the compound of formula 3E in the at least one reagent. In some embodiments, the contacting comprises heating at least one of the compound of formula 3E, the at least one reagent, or any combination thereof. In some embodiments, the contacting comprises cooling at least one of the compound of formula 3E, the at least one reagent, or any combination thereof.

In some embodiments, the contacting proceeds at a temperature of 0° C. to 150° C. For example, in some embodiments, the contacting proceeds at a temperature of 0° C. to 140° C., 0° C. to 130° C., 0° C. to 120° C., 0° C. to 110° C., 0° C. to 100° C., 0° C. to 90° C., 0° C. to 80° C., 0° C. to 70° C., 0° C. to 60° C., 0° C. to 50° C., 0° C. to 40° C., 0° C. to 30° C., 0° C. to 20° C., 0° C. to 10° C., 10° C. to 150° C., 20° C. to 150° C., 30° C. to 150° C., 40° C. to 150° C., 50° C. to 150° C., 60° C. to 150° C., 70° C. to 150° C., 80° C. to 150° C., 90° C. to 150° C., 100° C. to 150° C., 110° C. to 150° C., 120° C. to 150° C., 130° C. to 150° C., or 140° C. to 150° C. In some embodiments, the contacting proceeds at a temperature of 50° C. to 150° C., 60° C. to 140° C., 70° C. to 130° C., or 80° C. to 120° C.

In some embodiments, the at least one reagent comprises at least one of a metal tert-butoxide compound, a tetrahydrofuran, or any combination thereof.

Some embodiments relate to a composition. In some embodiments, the composition comprises at least one of any one or more of the compounds disclosed herein, the reaction products disclosed herein, or any combination thereof. In some embodiments, the composition comprises at least one of the compounds disclosed herein at a purity of at least 95%, at least 96%, at least 97%, at least 98%, at least 99%, at least 99.9%, at least 99.99%, at least 99.999%. For example, in some embodiments, the compound has a purity of 95% to 99.9999%, 95% to 99.999%, 95% to 99.99%, 95% to 99.9%, 95% to 99%, 95% to 98%, 95% to 97%, 95% to 96%, 96% to 99.9999%, 97% to 99.9999%, 98% to 99.9999%, 99% to 99.9999%, 99.9% to 99.9999%, 99.99% to 99.9999%, or 99.999% to 99.9999%, or any range or subrange between 95% and 99.9999%.

In some embodiments, the composition comprises at least one of the reaction products disclosed herein at a purity of at least 95%, at least 96%, at least 97%, at least 98%, at least 99%, at least 99.9%, at least 99.99%, at least 99.999%. For example, in some embodiments, the reaction product has a purity of 95% to 99.9999%, 95% to 99.999%, 95% to 99.99%, 95% to 99.9%, 95% to 99%, 95% to 98%, 95% to 97%, 95% to 96%, 96% to 99.9999%, 97% to 99.9999%, 98% to 99.9999%, 99% to 99.9999%, 99.9% to 99.9999%, 99.99% to 99.9999%, or 99.999% to 99.9999%, or any range or subrange between 95% and 99.9999%.

Any one or more of the embodiments disclosed herein shall be understood to be combinable without departing from the scope or spirit of the disclosure.

### Example 1

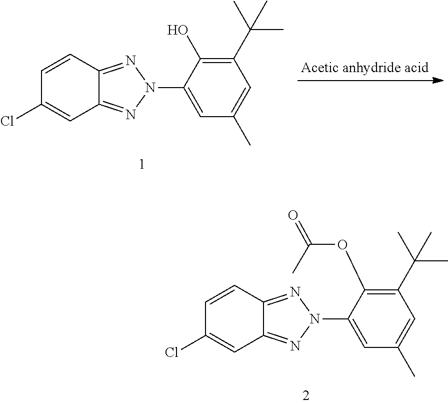 [SMILES: Cc1cc(-n2nc3ccc(Cl)cc3n2)c(O)c(C(C)(C)C)c1 ; CC(=O)Oc1c(-n2nc3ccc(Cl)cc3n2)cc(C)cc1C(C)(C)C]

As the first step, 100 g (0.32 mol) of compound 1 was suspended in 240 g (2.36 mol) of acetic anhydride and 5 g of concentrated H₂SO₄. The reaction mixture was stirred at room temperature for 10 minutes and heated at 100° C. for 2 h. The reaction mixture was poured slowly into a crushed ice (˜750 g) containing beaker while stirring with a glass rod. The resulting product was isolated by suction filtration. The crude product recrystallized in methanol/DCM and resulted in 104 g of pale-yellow product 2 with 89% yield and 99.9% purity.

### Example 2

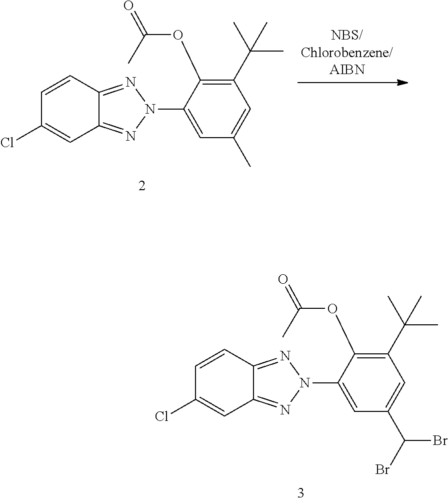 [SMILES: CC(=O)Oc1c(-n2nc3ccc(Cl)cc3n2)cc(C)cc1C(C)(C)C ; CC(=O)Oc1c(-n2nc3ccc(Cl)cc3n2)cc(C(Br)Br)cc1C(C)(C)C]

For the second step, 100 g (0.28 mol) of compound 2, 10 g (61 mmol) of azobisisobutyronitrile (AIBN), 109 g (0.62 mol) of N-bromosuccinimide (NBS), and 1 L chlorobenzene were charged into a 3 L flask under nitrogen. The reaction mixture was heated to 110° C. for 24 h. Then all the solvents were evaporated, and the crude mixture was dissolved in 250 mL DCM. The DCM solution was washed with 1 L water and dried with anhydrous MgSO₄. The DCM solution was filtered, and all the volatiles were removed. The crude product recrystallized in methanol/DCM and was dried under vacuum to result in 65.7 g of pale-yellow product 3 with 53% yield and 98.2% purity.

### Example 3

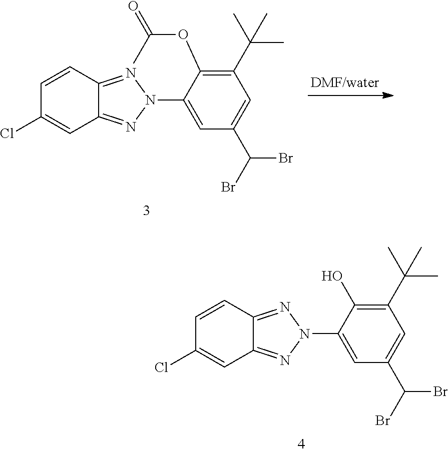 [SMILES: CC(=O)Oc1c(-n2nc3ccc(Cl)cc3n2)cc(C(Br)Br)cc1C(C)(C)C ; CC(C)(C)c1cc(C(Br)Br)cc(-n2nc3ccc(Cl)cc3n2)c1O]

For the third step, 20 g (39 mmol) of compound 3, 100 g of DMF and 1.5 mL of water were charged in a 1 L flask. The reaction mixture was heated to 120° C. for 6 h. Then the resulting mixture allowed to cool <80° C. and added to 500 g crushed ice containing with stirring with glass rod. The product isolated by suction filtration. The crude product recrystallized in methanol/DCM resulted 10.9 g of pale-yellow product 4 with 85% yield and 97.4% purity.

### Example 4

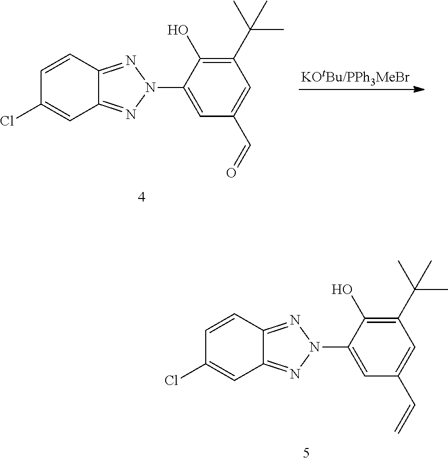 [SMILES: CC(C)(C)c1cc(C=O)cc(-n2nc3ccc(Cl)cc3n2)c1O ; C=Cc1cc(-n2nc3ccc(Cl)cc3n2)c(O)c(C(C)(C)C)c1]

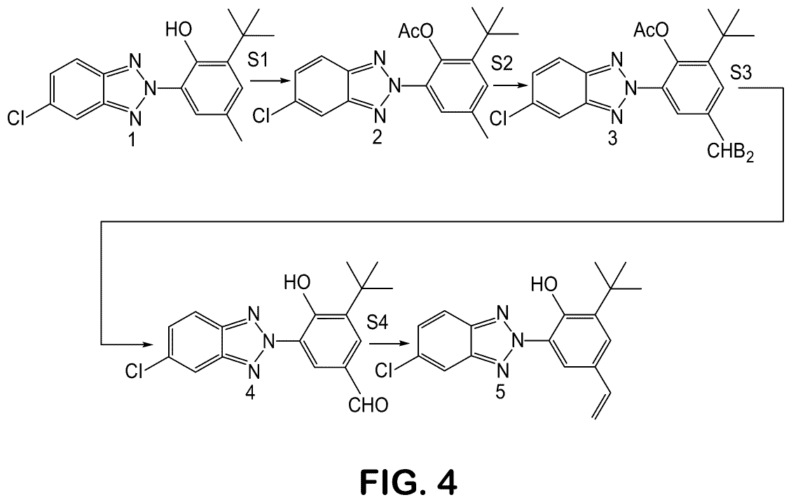

For the fourth step, 10.7 g (30 mmol) of PPh₃MeBr, 3.4 g (30 mmol) of KO^{t}Bu, and 100 g of THF were charged in a 500 mL flask. The reaction mixture was stirred at room for 24 h. 5 g of compound 4 was added to the above reaction mixture and stirred for 18 h. Then, all the volatiles were removed, and the crude material dissolved in 100 mL DCM and 100 mL cold water added. Organic layer separated and passed through celite. All the volatiles were removed. The crude product recrystallized in methanol/DCM resulted 1.8 g of pale-yellow product 5 with 40% yield and 96.2% purity. FIG. 4 is a schematic diagram of a reaction scheme illustrating Examples 1-4, according to some embodiments.

### Example 5

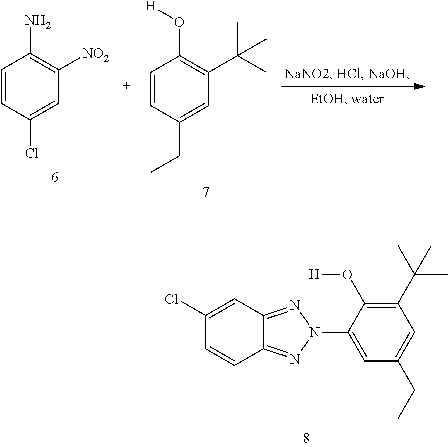 [SMILES: Nc1ccc(Cl)cc1[N+](=O)[O-] ; CCc1ccc(O)c(C(C)(C)C)c1 ; CCc1cc(-n2nc3ccc(Cl)cc3n2)c(O)c(C(C)(C)C)c1]

As the first step, 100 g (0.57 mol) of compound 6 was charged in 2 L flask and 300 g concentrated HCl was added to the above flask. The resulting mixture was heated at 70° C. for 1 h. Then, the resulting mixture was cooled to 0° C. to 5° C. Then, 79 g (1.15 mol) of NaNO₂ was dissolved in 200 ml DI water in a beaker. The NaNO₃ solution was added dropwise to the 2 L flask under vigorous stirring while maintaining the temperature 0° C. to 5° C. The resulting mixture was maintained at that temperature for 1 h, and 0.5 g of thiourea was added to eliminate the excess/unreacted nitrous acid. The above solution was added dropwise to a 5 L flak containing 102 g (0.57 mol) compound 7 and 2 L 1M HCl under vigorous stirring. The resulting mixture was then stirred for another 1 h. Then, the azo compound was allowed to precipitate and decant the mother liquor (ML). The product was washed with cold water 2 times and the material was dried in an oven at room temperature under vacuum overnight. This resulted in 171 g crude material. The azo compound was dissolved in 2 L ethanol and 2 L 2M NaOH solution. Then, 110 g of Zn dust was added portion-wise for a period of 1 h to the mixture and refluxed for 6 h. The resulting mixture was then filtrated and neutralized with concentrated HCl to result in a precipitate. The precipitate was isolated by filtration and recrystallized in DCM/Methanol to result in 108 g of compound 8 with 57.2% yield and 99.9% purity.

### Example 6

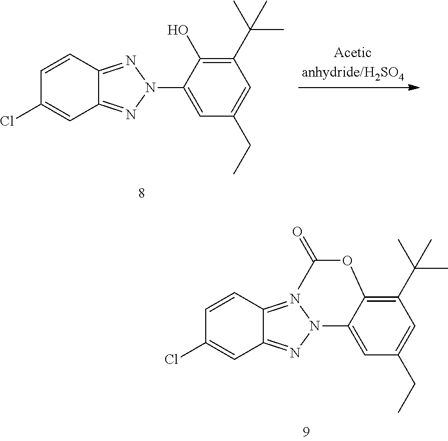 [SMILES: CCc1cc(-n2nc3ccc(Cl)cc3n2)c(O)c(C(C)(C)C)c1 ; CCc1cc(-n2nc3ccc(Cl)cc3n2)c(OC(C)=O)c(C(C)(C)C)c1]

For the second step, 60 g (0.18 mol) of compound 8 was suspended in 200 g (2 mol) of acetic anhydride and 3 g of concentrated H₂SO₄. The reaction mixture was stirred at room temperature for 30 minutes and heated at 100° C. for 2 h. The reaction mixture was poured slowly into a crashed ice (˜500 g) containing beaker while stirring with glass rod. The resulting product was isolated by suction filtration. The crude product was recrystallized in methanol/DCM and resulted in 64.8 g of pale-yellow product 9 with 96% yield and 99.7% purity.

### Example 7

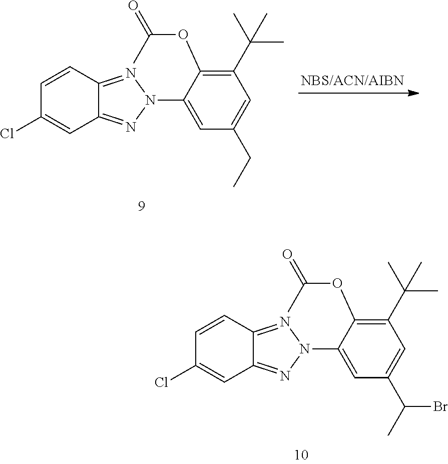 [SMILES: CCc1cc(-n2nc3ccc(Cl)cc3n2)c(OC(C)=O)c(C(C)(C)C)c1 ; CC(=O)Oc1c(-n2nc3ccc(Cl)cc3n2)cc(C(C)Br)cc1C(C)(C)C]

For the third step, 20 g (54 mmol) of compound 2, 0.88 g (5.4 mmol) of an azobisisobutyronitrile (AIBN), 10 g (59 mmol) of a N-bromosuccinimide (NBS) and 300 mL acetonitrile were charged in a 1 L flask under nitrogen. The reaction mixture was heated to 75° C. for 4 h. Then all the solvents evaporated, and the crude mixture was dissolved in 300 mL DCM. The DCM solution was washed with 1 L water and dried with anhydrous MgSO₄. The DCM solution passed through celite. All the volatiles were evaporated. The resulting rude product was recrystallized in methanol/DCM and dried under vacuum resulted 21.1 g of pale-yellow product 10 with 88% yield and 99.7% purity.

### Example 8

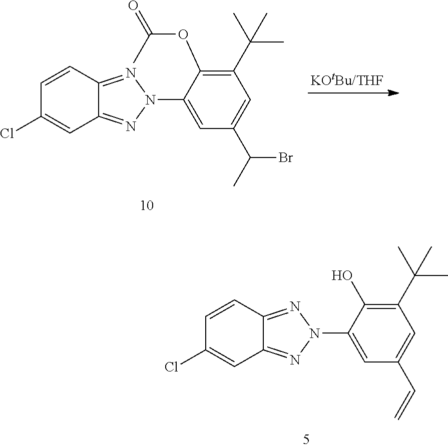 [SMILES: CC(=O)Oc1c(-n2nc3ccc(Cl)cc3n2)cc(C(C)Br)cc1C(C)(C)C ; C=Cc1cc(-n2nc3ccc(Cl)cc3n2)c(O)c(C(C)(C)C)c1]

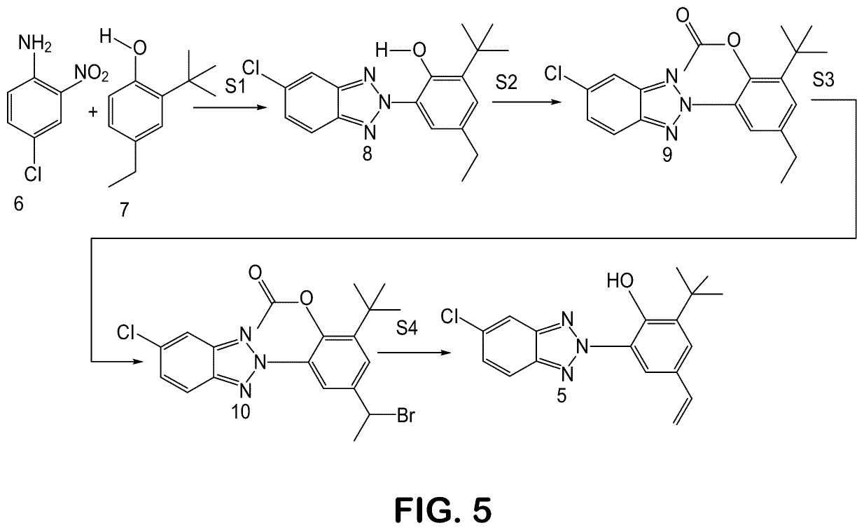

For this step, 20 g (44.4 mmol) of 10 was dissolved in 270 mL of THF in a 1 L flask. The reaction mixture cooled to 8-10° C. and 10.5 g (93.2 mmol) KOtBu added. The resulting mixture allowed to warm to room temperature and stirred at room for 2 h. Then, the resulting mixture cooled to 5'C. 100 mL of DCM and 300 ml of water were added to the above mixture. The organic phase was isolated, and all the volatiles were removed. The crude product recrystallized in methanol/DCM resulted 14.1 g of pale-yellow product 5 with 95% yield and 98% purity. FIG. 5 is a schematic diagram of a reaction scheme illustrating Examples 5-8, according to some embodiments.

### ASPECTS

Various Aspects are described below. It is to be understood that any one or more of the features recited in the following Aspect(s) can be combined with any one or more other Aspect(s).

Aspect 1. A method comprising:
obtaining a compound of the formula:

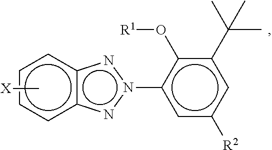

where:
   X is a first halide;
   R¹ is a first substituent,
   R² is a second substituent,
      wherein the second substituent comprises a second halide or an aldehyde; and
         contacting the compound with at least one reagent sufficient to obtain a reaction product of the formula:

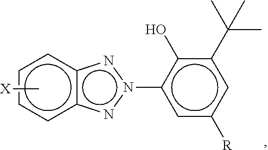

where:
   X is the first halide;
   R is a third substituent,
      wherein the third substituent is different from the second substituent.
         Aspect 2. The method according to Aspect 1, wherein R¹ is a substituent of the formula:

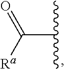

where:
   R^{a} is a C₁-C₄ alkyl.
      Aspect 3. The method according to any one of Aspects 1-2, wherein R² is a substituent of the formula:

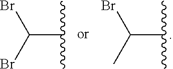

Aspect 4. The method according to any one of Aspects 1-3, wherein R² is a substituent of the formula-CHO.
Aspect 5. The method according to any one of Aspects 1-4, wherein the at least one reagent comprises at least one of a dimethylformamide (DMF), a water, a metal tert-butoxide, a tetrahydrofuran (THF), a PPh₃(CH₃)Br compound, or any combination thereof.
Aspect 6. The method according to any one of Aspects 1-5, wherein the at least one reagent comprises a potassium tert-butoxide compound and a tetrahydrofuran.
Aspect 7. The method according to Aspect 6, wherein the at least one reagent further comprises a PPh₃(CH₃)Br compound.
Aspect 8. The method according to any one of Aspects 1-7, wherein the contacting proceeds in a single step.
Aspect 9. The method according to any one of Aspects 1-8, wherein the contacting proceeds at a temperature of 0° C. to 150° C.
Aspect 10. The method according to any one of Aspects 1-9, wherein the reaction product is a compound of the formula:

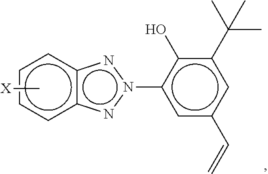

where:
   X is Cl, Br, F, or I.
      Aspect 11. The method according to Aspect 10, wherein X is Cl.
      Aspect 12. The method according to any one of Aspects 1-11, further comprising recovering the reaction product at a purity of at least 97%.
      Aspect 13. The method according to any one of Aspects 1-12, wherein the third substituent is not an alkyne.
      Aspect 14. The method according to any one of Aspects 1-13, wherein the method does not comprise CCl₄.
      Aspect 15. The method according to any one of Aspects 1-14, wherein the method does not comprise AgNO₃.
      Aspect 16. A method comprising:

obtaining a compound of the formula:

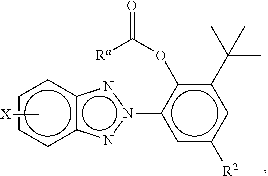

where:
   X is a first halide;
   R^{a} is a C₁-C₄ alkyl;
   R² comprises a second halide; and
      contacting the compound with at least one reagent sufficient to obtain a reaction product of the formula:

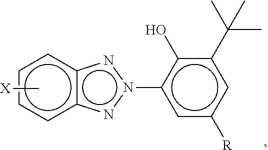

where:
   X is the first halide;
   R is an alkenyl.
      Aspect 17. The method according to Aspect 16, wherein the at least one reagent comprises at least one of a metal tert-butoxide compound, a tetrahydrofuran, or any combination thereof.
      Aspect 18. A method comprising:
obtaining a compound of the formula:

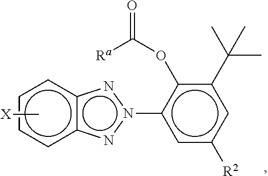

where:
   X is a first halide;
   R^{a} is a C₁-C₄ alkyl;
   R² comprises an aldehyde; and
      contacting the compound with at least one reagent sufficient to obtain a reaction product of the formula:

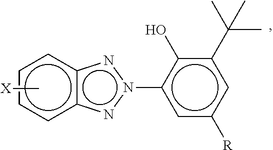

where:
   X is the first halide;
   R is an alkenyl.
      Aspect 19. The method according to Aspect 18, wherein the at least one reagent comprises at least one of a metal tert-butoxide compound, a tetrahydrofuran, or any combination thereof.
      Aspect 20. The method according to any one of Aspects 18-19, wherein the at least one reagent comprises at least one of a metal tert-butoxide compound, a tetrahydrofuran, a PPh₃(CH₃)Br compound, or any combination thereof.

What is claimed is:

1. A method comprising:
obtaining a compound of the formula: 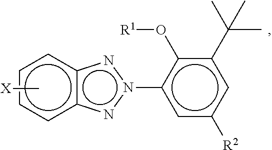
where:
X is a first halide;
R¹ is a first substituent;
R² is a second substituent,
wherein the second substituent comprises a second halide or an aldehyde; and
contacting the compound with at least one reagent sufficient to obtain a reaction product of the formula: 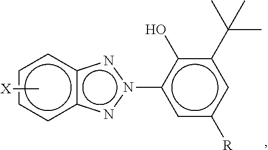
where:
X is the first halide;
R is a third substituent,
wherein the third substituent is different from the second substituent.

2. The method of claim 1, wherein R¹ is a substituent of the formula: 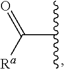
where:
R^{a} is a C₁-C₄ alkyl.

3. The method of claim 1, wherein R² is a substituent of the formula:

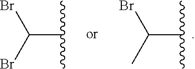

4. The method of claim 1, wherein R² is a substituent of the formula-CHO.

5. The method of claim 1, wherein the at least one reagent comprises at least one of a dimethylformamide (DMF), a water, a metal tert-butoxide, a tetrahydrofuran (THF), a PPh₃(CH₃)Br compound, or any combination thereof.

6. The method of claim 1, wherein the at least one reagent comprises a potassium tert-butoxide compound and a tetrahydrofuran.

7. The method of claim 6, wherein the at least one reagent further comprises a PPh₃(CH₃)Br compound.

8. The method of claim 1, wherein the contacting proceeds in a single step.

9. The method of claim 1, wherein the contacting proceeds at a temperature of 0° C. to 150° C.

10. The method of claim 1, wherein the reaction product is a compound of the formula: 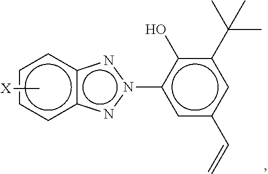
where:
X is Cl, Br, F, or I.

11. The method of claim 10, wherein X is Cl.

12. The method of claim 1, further comprising recovering the reaction product at a purity of at least 97%.

13. The method of claim 1, wherein the third substituent is not an alkyne.

14. The method of claim 1, wherein the method does not comprise CCl₄.

15. The method of claim 1, wherein the method does not comprise AgNO₃.

16. A method comprising:
obtaining a compound of the formula: 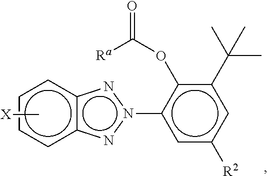
where:
X is a first halide;
R^{a} is a C₁-C₄ alkyl;
R² comprises a second halide; and
contacting the compound with at least one reagent sufficient to obtain a reaction product of the formula: 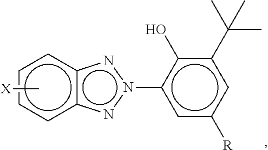
where:
X is the first halide;
R is an alkenyl.

17. The method of claim 16, wherein the at least one reagent comprises at least one of a metal tert-butoxide compound, a tetrahydrofuran, or any combination thereof.

18. A method comprising:
obtaining a compound of the formula: 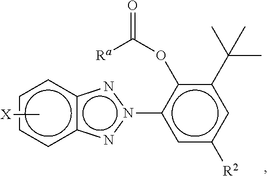
where:
X is a first halide;
R^{a} is a C₁-C₄ alkyl;
R² comprises an aldehyde; and
contacting the compound with at least one reagent sufficient to obtain a reaction product of the formula: 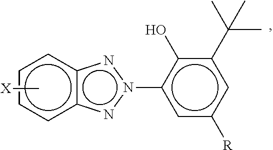
where:
X is the first halide;
R is an alkenyl.

19. The method of claim 18, wherein the at least one reagent comprises at least one of a metal tert-butoxide compound, a tetrahydrofuran, or any combination thereof.

20. The method of claim 18, wherein the at least one reagent comprises at least one of a metal tert-butoxide compound, a tetrahydrofuran, a PPh₃(CH₃)Br compound, or any combination thereof.
